SEQUENCE LISTING (36 sequences)

SEQ ID NO: 1 - DNA, 23 bp, synthetic construct
  source 1..23: mol_type = other DNA
ctcctaaagcagaagaggatcac

SEQ ID NO: 2 - DNA, 18 bp, synthetic construct
  source 1..18: mol_type = other DNA
tgagctgcaaagcctcaa

SEQ ID NO: 3 - DNA, 24 bp, synthetic construct
  source 1..24: mol_type = other DNA
ttcagccaaacgactatctgccgt

SEQ ID NO: 4 - DNA, 22 bp, synthetic construct
  source 1..22: mol_type = other DNA
gcagaaatacctcagtctccag

SEQ ID NO: 5 - DNA, 20 bp, synthetic construct
  source 1..20: mol_type = other DNA
ctattcctcggccagttgtt

SEQ ID NO: 6 - DNA, 24 bp, synthetic construct
  source 1..24: mol_type = other DNA
ttctggaaccaggtcttcacctgc

SEQ ID NO: 7 - DNA, 21 bp, synthetic construct
  source 1..21: mol_type = other DNA
cgtctccttcagcaaagtcaa

SEQ ID NO: 8 - DNA, 22 bp, synthetic construct
  source 1..22: mol_type = other DNA
gatgtggattcgaggctcatac

SEQ ID NO: 9 - DNA, 27 bp, synthetic construct
  source 1..27: mol_type = other DNA
tgtaaggagttcaacatgatctggccc

SEQ ID NO: 10 - DNA, 22 bp, synthetic construct
  source 1..22: mol_type = other DNA
ctgcacaccctcaggaattatc

SEQ ID NO: 11 - DNA, 21 bp, synthetic construct
  source 1..21: mol_type = other DNA
tccgtgagctctccgatatac

SEQ ID NO: 12 - DNA, 24 bp, synthetic construct
  source 1..24: mol_type = other DNA
ttcagcgtctggatcttggtgagc

SEQ ID NO: 13 - DNA, 18 bp, synthetic construct
  source 1..18: mol_type = other DNA
gggagaatggccgaaact

SEQ ID NO: 14 - DNA, 19 bp, synthetic construct
  source 1..19: mol_type = other DNA
gcatctccactcccatagc

SEQ ID NO: 15 - DNA, 21 bp, synthetic construct
  source 1..21: mol_type = other DNA
taagagggagcgagatgcccg

SEQ ID NO: 16 - DNA, 22 bp, synthetic construct
  source 1..22: mol_type = other DNA
gatcctttgcaggtatcaggag

SEQ ID NO: 17 - DNA, 21 bp, synthetic construct
  source 1..21: mol_type = other DNA
tctcgaatttcccagctgaac

SEQ ID NO: 18 - DNA, 22 bp, synthetic construct
  source 1..22: mol_type = other DNA
agcaagcccaagcaggtgacaa

SEQ ID NO: 19 - DNA, 22 bp, synthetic construct
  source 1..22: mol_type = other DNA
ctgcacaccctcaggaattatc

SEQ ID NO: 20 - DNA, 21 bp, synthetic construct
  source 1..21: mol_type = other DNA
tccgtgagctctccgatatac

SEQ ID NO: 21 - DNA, 24 bp, synthetic construct
  source 1..24: mol_type = other DNA
ttcagcgtctggatcttggtgagc

SEQ ID NO: 22 - DNA, 20 bp, synthetic construct
  source 1..20: mol_type = other DNA
ggcatgtttagctgggagat

SEQ ID NO: 23 - DNA, 22 bp, synthetic construct
  source 1..22: mol_type = other DNA
ctccttcttcccgaacttgatt

SEQ ID NO: 24 - DNA, 20 bp, synthetic construct
  source 1..20: mol_type = other DNA
accgtgccctcagtgagttc

SEQ ID NO: 25 - DNA, 20 bp, synthetic construct
  source 1..20: mol_type = other DNA
ccaggtcagcaactggttta

SEQ ID NO: 26 - DNA, 22 bp, synthetic construct
  source 1..22: mol_type = other DNA
gcttgttggaggaggagttatt

SEQ ID NO: 27 - DNA, 24 bp, synthetic construct
  source 1..24: mol_type = other DNA
aggccaaggaaagggagaacaccg

SEQ ID NO: 28 - DNA, 20 bp, synthetic construct
  source 1..20: mol_type = other DNA
gacaaggtgctgctcaagta

SEQ ID NO: 29 - DNA, 22 bp, synthetic construct
  source 1..22: mol_type = other DNA
ctgtgacctactgaatcgtctg

SEQ ID NO: 30 - DNA, 25 bp, synthetic construct
  source 1..25: mol_type = other DNA
cgtggagacgttgagaaagaagggc

SEQ ID NO: 31 - DNA, 17 bp, synthetic construct
  source 1..17: mol_type = other DNA
cagcggcgaacactaca

SEQ ID NO: 32 - DNA, 21 bp, synthetic construct
  source 1..21: mol_type = other DNA
ctgtagtaagtgcggtcgtag

SEQ ID NO: 33 - DNA, 24 bp, synthetic construct
  source 1..24: mol_type = other DNA
ttccgacggcatgatggactacag

SEQ ID NO: 34 - DNA, 20 bp, synthetic construct
  source 1..20: mol_type = other DNA
cagtgaatgcagctcccata

SEQ ID NO: 35 - DNA, 19 bp, synthetic construct
  source 1..19: mol_type = other DNA
gattgtgggcgtctgtagg

SEQ ID NO: 36 - DNA, 21 bp, synthetic construct
  source 1..21: mol_type = other DNA
caacccaggggatcatctgct

(19) United States
(12) Patent Application Publication
(10) Pub. No.: US 2024/0360415 A1
(43) Pub. Date: Oct. 31, 2024

(54) MUSCLE CELLS DIFFERENTIATED FROM PLURIPOTENT CELLS, METHODS OF PRODUCING SAME AND USE THEREOF

(71) Applicant: ALEPH FARMS LTD., Rehovot (IL)

(72) Inventors: Neta Lavon, Ness Ziona (IL); Natali Molotski-Handelman, Givataim (IL); Aviv Rom, Pardesia (IL)

(21) Appl. No.: 18/579,499

(22) PCT Filed: Aug. 8, 2022
(86) PCT No.: PCT/IL2022/050861

§ 371 Date: Jan. 15, 2024

Related U.S. Application Data
(60) Provisional application No. 63/230,849, filed on Aug. 9, 2021.

(51) Int. Cl. C12N 5/077; A23L 13/40; C12N 5/00

(52) U.S. Cl. CPC C12N 5/0658; A23L 13/45; C12N 5/0068; C12N 2501/15; C12N 2501/155; C12N 2501/19; C12N 2501/999; C12N 2506/1384; C12N 2506/45; C12N 2513/00

(57) ABSTRACT

The present invention relates to the field of production of bioengineered tissues comprising skeletal muscle cells, particularly to compositions and methods for producing a plurality of skeletal muscle-committed progenitor cells from pluripotent stem cells and skeletal muscle cells differentiated therefrom. The present invention further provides a mass of skeletal muscle-committed progenitor cells and/or a mass of skeletal muscle cells, engineered tissue comprising same, and uses thereof.

### FIELD OF THE INVENTION

The present invention relates to the field of production of bioengineered tissues comprising skeletal muscle cells, particularly to compositions and methods for producing a plurality of skeletal muscle-committed progenitor cells from pluripotent stem cells and skeletal muscle cells differentiated therefrom. The present invention further provides a mass of skeletal muscle-committed progenitor cells and/or a mass of skeletal muscle cells, engineered tissue comprising same and uses thereof.

### BACKGROUND OF THE INVENTION

Animal meat is a good nutritional source of protein, containing of all the essential amino acids in adequate proportions needed for growth and maintenance of the human body. However, modern practices of growing livestock for food have hazardous effects on air and water quality, and requires large land areas and energy investment. It also raises moral issue, as typically the livestock are grown in crowded habitats and sometimes inappropriate conditions afflicted upon the livestock subjects. For this reason, cell cultured meat products might also be potentially consumed by people who abstains meat for humanitarian reasons.

Among others, a challenge in producing cultured meat products is the texture and mouth-feel that fail to replicate those of equivalent slaughtered-meat products. Cultured meat containing cultured cells only is typically in a form of a ground meat, which significantly limits the variety of food that may be offered to consumers. While hybrid products, containing cultured cells and plant-based proteins form one potential solution, there is still a need to obtain a cell-based portion that mimics slaughtered meat, which is mainly composed of muscles.

Attempt for in vitro muscle differentiation have been taken mainly in basic science research and in the area of therapeutics development. The skeletal muscle lineage derives from the embryonic paraxial mesoderm (PM) which also gives rise to the axial skeleton, the dermis, brown fat, meninges, and endothelial cells. Experimental strategies recapitulating myogenesis in vitro from mouse and human pluripotent stem cells (embryonic stem cell or induced pluripotent stem cells) have recently been reported and all rely on early activation of Wnt signaling agents (such as CHIR) at the epiblast stage. This leads to induction of neuro-mesodermal progenitors (NMPs) that can subsequently be induced to a PM fate and to skeletal muscle. These protocols can efficiently produce fetal muscle fibers and immature satellite cells. Until recently, the only efficient protocols allowing differentiation of reasonably mature muscle cells relied on overexpression of transcription factors such as MyoD or Pax3/7 followed by cell sorting of the induced progenitors (Pourquié O et al., 2018. Curr Top Dev Biol. 129:123-142. doi: 10.1016/bs.ctdb.2018.03.003).

Prior studies indicated that Activin/Nodal/TGFβ, BMP, FGF, and WNT broadly induce mesoderm from pluripotent stem cells (Kyle M L et al., 2016. Cell 166(2):451-467). FGF and Activin were shown to be essential for cardiac differentiation (Shen M et al., 2021. Circulation Research 128:670-686; Sasano Y et al., 2020. Journal of Bioscience and Bioengineering, 129(6):749-755), while Activin A was shown as a negative regulator of muscle mass (Latres E et al., 2017. Nat Commun 8:15153, DOI: 10.1038/ncomms15153). It was shown that targeting (inhibiting) Activin A signaling pathway has significant beneficial effects in protecting against both muscle and bone loss in microgravity, suggesting that this strategy may be effective in preventing or treating muscle and bone loss (Lee Se-Jin et al., 2020. PNAS 117(38):23942-23951, doi.org/10.1073/pnas.2014716117). TGFβ inhibitor SB431542, a known somatic mesoderm-like cells inducer was shown to enhance myotube generation in the context of PAX7-induced myogenic differentiation (Selvaraj S et al., 2019. eLife 8:e47970, DOI: 10.7554/eLife.47970).

U.S. Application Publication No. 2012/0164731 discloses a method of producing skeletal muscle progenitor cells using pluripotent stem cells, particularly induced pluripotent cells, comprising obtaining PDGFRα-positive mesodermal cell by culturing pluripotent stem cells in the presence of Activin A, and thereafter culturing the obtained mesodermal cells under serum-free conditions and in the presence of a Wnt signal inducer, to allow the cell to differentiate into skeletal muscle progenitor cells. Also disclosed are a cell population containing the skeletal muscle progenitor cells as obtained by the method, and a skeletal muscle regeneration promoting agent and therapeutic agent for muscular diseases such as muscular dystrophy, the promoting agent or agent comprising the skeletal muscle progenitor cell as an active ingredient.

U.S. Application Publication No. 2019/0010460 discloses method for producing bioengineered heart muscle (BHM) from pluripotent stem cells, generally comprising the steps of inducing mesoderm differentiation, cardiac differentiation, and cardiac maturation by directed tissue formation.

Most of the current methods for differentiating muscle cells from pluripotent cells require the use of a large number of components, including costly growth factors and media and further require long culturing period.

There is a need for a simple and economic compositions and methods for obtaining muscle cells that may be used in the industry of cell cultured products, including cultured meat products.

### SUMMARY OF THE INVENTION

The present invention answers the above-described need for a mass of skeletal muscle cells that may form part of engineered tissues or cultured meat products, providing compositions and methods for producing a mass of skeletal muscle-committed progenitor cells that may easily be further differentiated to muscle cells.

The present invention is based in part on the unexpected discovery that pluripotent stem cells (PSCs) cultured in a medium comprising a combination of an activator of the TGF-beta (TGF-β) signaling pathway, particularly Activin A, and an inhibitor of the Glycogen synthase kinase-3 (GSK3) signaling pathway, particularly CHIR-99021, produces a plurality of cells comprising skeletal muscle-committed progenitor cells. Said muscle-committed progenitor cells are then differentiated into muscle cells. Advantageously, the plurality of cells further comprises additional lineage-committed cells, that can also easily differentiate to extracellular-matrix (ECM)-producing cells and to adipocytes.

Moreover, the teachings of the present invention are advantageous over previously known methods for producing skeletal muscle-committed progenitor cells from PSCs in that the lineage commitment differentiation process requires only a medium supplemented with nutrients and the combination of at least one TGF-β activator and at least one GSK3 inhibitor. Also, the methods of the present invention enable to shorten the production of muscle cells from PSCs to a time frame of days, and completing the entire process of producing bioengineered tissue comprising skeletal muscle cells in about two- to about four weeks. Furthermore, the method is easily scalable to reactors in a volume of tens liters, which enables its use in the food industry at costs comparable to those of traditional processes in this industry.

According to certain aspects, the present invention provides a method of producing a plurality of cells comprising skeletal muscle-committed progenitor cells, the method comprising culturing a plurality of pluripotent stem cells (PSCs) in a culture medium comprising a combination of: (i) at least one activator of the TGF-beta (TGF-β) signaling pathway and (ii) at least one inhibitor of the GSK3 signaling pathway, thereby producing a plurality of cells comprising skeletal muscle-committed progenitor cells.

According to certain embodiments, the medium is a serum-free medium.

According to certain embodiments, culturing is performed under three-dimensional (3D) culture conditions. According to certain exemplary embodiments, the 3D culture is a suspension culture. According to certain embodiments, the suspension culture is devoid of adherent material and/or support matrix. According to these embodiments, the cells are self-assembled forming at least one cell aggregate. According to certain embodiments, the cell aggregate is in a form selected from the group consisting of a cluster, a spheroid, an organoid and the like.

According to yet additional or alternative embodiments, the suspension culture comprises at least one adherent material and/or support matrix. According to certain exemplary embodiments, the 3D culturing is performed within a vessel.

According to certain alternative embodiments, culturing is performed under two-dimensional (2D) culture conditions. According to some embodiments, the 2D culture comprises at least one adherent material and/or support matrix.

Any adherent material or a support matrix known or to be known in the art that enables and supports cell culturing, particularly culturing of PSCs and cell differentiated therefrom may be used according to the teachings of the present invention. According to certain embodiments, the support matrix is a semi-solid matrix. According to some embodiments, the support matrix is a solid.

According to certain embodiments, the at least one activator of the TGF-β signaling pathway is selected from the group consisting of: Activin A, TGF-β, BMP2, BMP7, GDF9, NODAL, and any combination thereof. Each possibility represents a separate embodiment of the present invention.

According to certain embodiments, the inhibitor of the GSK3 signaling pathway is selected from the group consisting of: CHIR-99021 (C22H18C12N8) or a salt thereof, SB 216763, LY2090314, TWS119, Tideglusib, GSK-3β inhibitor 1, GSK-3β inhibitor 2, GSK-3β inhibitor 3, AR-A014418, TDZD-8, Kenpaullone, GSK3 Inhibitor IX, Cromolyn sodium, CHIR-98014, AZD1080, SB 415286, IM-12, 9-ING-41, Indirubin-3′-monoxime, 1-Azakenpaullone, BRD0705, AZD2858, CP21R7, BIO-acetoxime, Bikinin, VP3.15, VP3.15 dihydrobromide, GNF4877, KY19382, SAR502250, A 1070722, (R)-BRD3731, BRD3731, BIP-135, 5-Iodo-indirubin-3′-monoxime, BRD5648, GSK3 inhibitor 1, GSK3/CDK5/CDK2-IN-1, Indirubin-3′-monoxime-5-sulphonic acid, a GSK3β-inhibiting flavonoid, lithium, and any combination thereof. Each possibility represents a separate embodiment of the present invention.

According to certain embodiments, the CHIR-99021 salt is selected from the group consisting of CHIR-99021 monohydrochloride and CHIR-99021 trihydrochloride. Each possibility represents a separate embodiment of the present invention.

According to certain embodiments, the GSK3β-inhibiting flavonoid is selected from the group consisting of luteolin, apigenin, quercetin, myricetin and any combination thereof. Each possibility represents a separate embodiment of the present invention.

According to certain embodiments, the medium is devoid of growth factors other than growth factors activating the TGF-β pathway. According to some embodiments, the medium is devoid of bFGF.

According to certain exemplary embodiments, the combination comprising at least one activator of the TGF-β signaling pathway and at least one inhibitor of GSK3 signaling pathway comprises Activin A and CHIR-99021. According to certain embodiments, the combination is consisting of Activin A and CHIR-99021.

According to certain embodiments, at the time of seeding, the medium further comprises an inhibitor of Rho-associated protein kinase (Rock).

According to certain embodiments, culturing is performed for a time period enabling to reach from about 10% to about 90% skeletal muscle-committed progenitor cells out of the total number of the plurality of cells.

According to certain embodiments, culturing the plurality of PSCs is performed continuously in the medium comprising the combination of at least one activator of the TGF-β signaling pathway and at least one inhibitor of the GSK3 signaling pathway.

According to certain additional or alternative embodiments, culturing the plurality of PSCs is performed in cycles, wherein the medium comprising the combination of at least one activator of the TGF-β signaling pathway and at least one inhibitor of the GSK3 signaling pathway is replaced after each cycle. The combination of at least one activator of the TGF-β signaling pathway and at least one inhibitor of the GSK3 signaling pathway in each cycle may be the same or different.

According to certain embodiments, the produced plurality of cells further comprises at least one additional lineage committed progenitor cells. According to certain embodiments, the at least one additional lineage-committed progenitor cells are selected from stromal-committed progenitor cells, adipocyte-committed progenitor cells and a combination thereof. Each possibility represents a separate embodiment of the present invention.

According to certain embodiments, the plurality PSCs is of an origin selected from the group consisting of non-human animal and human. According to certain embodiments, the non-human animal is selected from the group consisting of ungulate, poultry, aquatic animals, invertebrate and reptiles. According to certain embodiments, the ungulate is selected from the group consisting of a bovine, a sheep, a goat, a buffalo, an equine, a pig, a giraffe, a camel, a deer, a hippopotamus, or a rhinoceros. According to certain exemplary embodiments, the ungulate is a bovine.

According to certain embodiments, the PSCs are selected from the group consisting of induced PSCs (iPSCs), embryonic stem cells (ESCs) and non embryonic stem cells.

According to certain embodiments, the PSCs are not genetically modified.

According to certain embodiments, the PSCs are genetically modified.

According to another aspect, the present invention provides a method of producing a plurality of skeletal muscle cells, the method comprising:
depositing a plurality of cells comprising skeletal muscle-committed progenitor cells produced by the method of the invention as described hereinabove on an adherent material and/or support matrix; and
culturing the plurality of cells in a differentiation medium promoting differentiation of the skeletal muscle-committed progenitor cells to skeletal muscle cells,
thereby producing a plurality of differentiated cells comprising skeletal muscle cells.

Various differentiation medium types can be used with the method of the present invention. Nevertheless, the present invention now demonstrates that a serum-free medium supplemented with nutrients and optionally certain hormones suffices for obtaining differentiation of the muscle-committed progenitor cells produced by the methods of the invention to skeletal muscle cells. According to certain embodiments, the differentiation medium is devoid of an activator of the TGF-β signaling pathway and of an inhibitor the GSK3 signaling pathway. According to certain exemplary embodiments, the medium is devoid of Activin A and CHIR-99021.

According to certain embodiments, the cells are cultured in the differentiation medium for a period of from about 3 days to about 30 days. According to certain embodiments, the cells are cultured in the differentiation medium for from about 3 days to about 10 days. According to certain embodiments, the cells are cultured in the differentiation medium for from about 5 days to about 8 days. According to certain embodiments, the entire period for obtaining a plurality of differentiated cells comprising skeletal muscle cells is from about 6 days to about 30 days. According to certain embodiments, the entire period for obtaining a plurality of differentiated cells comprising skeletal muscle cells is from about 6 days to about 12 days.

According to certain embodiments, the plurality of differentiated cells comprises from about 10% to about 90% skeletal muscle cells out of the total number of cells.

According to certain embodiments, the skeletal muscle cells are viable.

The present invention further provides a plurality of cells comprising skeletal muscle-committed progenitor cells produced by the methods of the present invention. According to certain embodiments, the plurality of skeletal muscle-committed progenitor cells is essentially devoid of PSCs. As used herein, the term “essentially devoid” with regard to PSCs refers to a number of PSCs which is not detectable by standard methods currently known in the Art.

According to certain embodiments, the skeletal muscle-committed progenitor cells are produced from non-human animal PSCs. According to certain embodiments, the skeletal muscle-committed progenitor cells are produced from bovine PSCs.

The present invention further encompasses an engineered tissue comprising differentiated skeletal muscle cells produced by the methods of the present invention as described herein. According to certain embodiments, the differentiated muscle skeletal cells are produced from non-human animal PSCs. According to certain embodiments, the differentiated muscle cells are produced from bovine PSCs.

According to certain embodiments, the plurality of differentiated cells comprises from about 10% to about 90% differentiated skeletal muscle cells out of the total number of cells. According to certain embodiments, the plurality of differentiated cells further comprises at least one of differentiated stromal cells, differentiated adipocyte cells, and a combination thereof. According to certain embodiments, the differentiated stromal cells comprise from about 10% to about 90% out of the total number of cells. According to certain embodiments, the differentiated adipocyte cells comprise from about 10% to about 90% out of the total number of cells. According to certain embodiments, the stromal cells are ECM-producing cells. According to certain embodiments, the plurality of differentiated cells is devoid of cardiac muscle cells. The plurality of differentiated cells and/or engineered tissue comprising skeletal muscle cells of the present invention can be used in a variety of applications, mainly depending on the source and type of the PSCs from which the skeletal muscle cells are differentiated.

According to certain exemplary embodiments, the plurality of differentiated cells and/or engineered tissue comprising skeletal muscle cells of the present invention is used for the production of cultured food products, particularly of cultured meat.

According to yet further certain aspects, the present invention provides a plurality of in vitro grown cells comprising skeletal muscle-committed progenitor cells, wherein the skeletal muscle-committed progenitor cells are characterized by the expression of at least one mesodermal marker and/or at least one early myogenic marker.

According to certain embodiments, the at least one mesodermal marker is selected from the group consisting of TBXT, TBX6, MSGN1, Pax3 and any combination thereof and the at least one early myogenic marker is Six1. Each possibility represents a separate embodiment of the present invention.

According to certain embodiments, the skeletal muscle-committed progenitor cells are characterized by the expression of MSGN1 and Six1.

According to certain embodiments, the plurality of in vitro grown cells further comprises at least one additional lineage committed cells selected from the group consisting of stromal-committed progenitor cells, adipocyte-committed progenitor cells and a combination thereof. Each possibility represents a separate embodiment of the present invention.

According to certain embodiments, the plurality of in vitro grown cells comprises at least one GSK3β inhibiting flavonoid and/or a metabolite thereof.

According to additional certain aspects, the present invention provides a plurality of in vitro grown cells comprising differentiated skeletal muscle cells, wherein the skeletal muscle cells are characterized by the expression of at least one myogenic marker.

According to certain embodiments, the at least one myogenic marker is selected from the group consisting of Myf5, Pax7, MEF2C, SIX1, NYOD1, MYOG, MYH3, MYH7, NYH8, MB, MYMK and any combination thereof. Each possibility represents a separate embodiment of the present invention.

According to certain embodiments, the plurality of in vitro grown cells further comprises at least one of stromal cells, adipocytes or a combination thereof. Each possibility represents a separate embodiment of the present invention.

According to certain embodiments, the stromal cells are extracellular-matrix (ECM) producing cells.

According to certain embodiments, the cells within the plurality of the in vitro grown cells are non-human-animal cells.

The present invention further encompasses an engineered tissue comprising the plurality of in-vitro grown cells comprising skeletal muscle cells.

According to certain embodiments, the in-vitro grown differentiated cells comprising skeletal muscle cells are non-human animal cells. According to these embodiments, the plurality of cells or the engineered tissue comprising same form a cultured food product, particularly cultured meat product.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows a representative Bright Field image of skeletal muscle committed progenitor cells obtained from bovine pluripotent stem cells after 4 days incubation in a serum-free growth medium comprising a combination of Activin A and of CHIR-99021 in suspension. Magnification ×4.

FIG. 2 shows a representative RT-PCR analysis of markers after 4 days of differentiation starting from bovine PSCs incubated in a serum-free growth medium comprising Activin A and CHIR-99021.

FIG. 3 shows a representative immunofluorescence staining of Myosin heavy chain (using MF-20 antibody) after total of 11 days of differentiation starting from bovine PSCs. Bovine PSCs were cultured in a 3D suspension (FIGS. 3A-3C) or in a 2D culture on a support (FIG. 3D) for the first 4 days in a serum-free growth medium comprising CHIR-99021 only (FIG. 3A); serum-free growth medium comprising CHIR-99021 in combination with Activin A (FIGS. 3B and 3D); or serum-free growth medium comprising CHIR-99021 in combination with TGFβ (FIG. 3C). After 4 days, the cells/cell aggregates were deposited on a 2D support and cultured for additional 7 days in serum-free medium devoid of Activin A, CHIR-99021 and TGFβ. Grey—DAPI/nuclei; white—MyhC positive cells. Magnification ×10.

FIG. 4 shows a representative RT-PCR analysis of myogenic markers Myf5, Pax7, Mef2C, Six1, MyoD1, MyoG, MYH3, MYH7, MYH8, Myoglobin (MB) and Myomaker (MYMK) after total of 11 days of differentiation starting from bovine pluripotent stem cells incubated in a serum-free growth media comprising Activin A and CHIR-99021 only during the commitment stage, i.e., during the first 4 days.

### DETAILED DESCRIPTION OF THE INVENTION

The present invention provides methods for producing skeletal muscle cells from pluripotent stem cells (PSCs), which are suitable for large-scale, cost-effective production. The present invention is based in part on the unexpected discovery that culturing PSCs in the presence of a combination of Activin A, an activator of the TGF-β signaling pathway hitherto known as a negative regulator of muscle mass, and CHIR 99021, an inhibitor of the GSK3 signaling pathway, resulted in a significant number of PSCs converted to mesoderm lineage cells, which, in turn, can differentiate to skeletal muscle cells. Furthermore, the culturing media required throughout the process of producing a mass of a plurality of skeletal muscle cells and/or engineered tissue comprising same are simple to produce and comprise minimal number of growth factors and small molecules; the entire growth cycle is shorter comparing to hitherto known protocols; enabling large scale production at an economical cost, even when the products are to be used in the food industry.

The present invention further provides skeletal muscle cells produced by the methods of the invention, that can be used for therapeutic applications, in a form of plurality of cells or of an engineered tissue comprising same. In certain embodiments, the skeletal muscle cells and engineered tissues comprising same of the present invention are tailored for the food industry, particularly for the production of cultured meat.

### Definitions

As used herein, the singular form “a”, “an” and “the” include plural references unless the context clearly dictates otherwise. For example, the term “a compound” or “at least one compound” may include a plurality of compounds, including mixtures thereof.

The terms “comprise”, “comprising”, “includes”, “including”, “having” and their conjugates mean “including but not limited to”.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The term “about” as used herein refers to a variation of a numerical designation of +10% or −10% of the numerical designation. Moreover, all numerical ranges herein should be understood to include each whole integer within the range.

As used herein, the term “plurality”, particularly with reference to PSCs, refers to “at least two”, particularly at least two cells, at least 5 cells, at least 10 cells or at least 100, or at least, or at least 1,000, or at least 10,000 cells.

As used herein the term “pluripotent stem cells (PSCs)” refers to cells that can propagate indefinitely, as well as give rise to every other cell type in the body. The term explicitly comprises both naïve and primed pluripotent stem cells.

As used herein the term “induced pluripotent stem cells (iPSCs)” refers to a type of pluripotent stem cell that can be generated directly from somatic cells.

As used herein the term “embryonic stem cells (ESC)” refers to a type of pluripotent stem cell derived blastocyst.

As used herein, the term “engineered tissue” as used herein refers to association of cells in X and Y planes that is multiple cells thick, forming at least one layer. In some embodiments, the engineered tissue includes one layer. In other embodiments, the engineered tissue includes a plurality of layers. In some embodiments, a layer forms a contiguous, substantially contiguous, or non-contiguous sheet of cells. In some embodiments, the engineered tissue or a layer thereof comprises multiple cells in the X, Y, and Z axes. The engineered tissue according to the teachings of the present invention may or may not comprise a cell-adherent material and/or support matrix. According to some embodiments, the cell-adherent material and/or support matrix forms nanocarriers, microcarriers, macro-carriers or a combination thereof. According to some embodiments, the cell-adherent material and/or support matrix forms a scaffold.

The term “cultured meat” is used herein to describe meat grown from in vitro non-human animal cell culture distinguished from meat of slaughtered animals. Additional terms that may be used in the Art to describe meat grown from in vitro animal cell culture include cell grown meat, cell cultured meat, cultivated meat, clean meat, lab-grown meat, test tube meat, in vitro meat, tube steak, synthetic meat, tissue engineered meat, engineered meat, artificial meat, and manmade meat.

According to certain aspects, the present invention provides a method of producing a plurality of cells comprising muscle-committed progenitor cells, comprising culturing a plurality of pluripotent stem cells (PSCs) in a culture medium comprising a combination of: (i) at least one activator of the TGF-beta (TGF-β) signaling pathway and (ii) at least one inhibitor of the GSK3 signaling pathway; thereby producing a plurality of muscle-committed progenitor cells.

It is to be explicitly understood that both, the at least one activator of the TGF-β signaling pathway and the at least one inhibitor of the GSK3 signaling pathway are present within the culture medium for the entire incubation time until skeletal muscle-committed progenitor cells are formed.

TGF-β signaling is involved in many cellular functions, including cell growth, cell fate and apoptosis. Signaling typically begins with binding of a TGF-β superfamily ligand to a type II receptor which recruits and phosphorylates a type I receptor. The type I receptor then phosphorylates the SMAD family of transcription factors, which act as transcription factors in the nucleus and regulate target gene expression. The TGF-β superfamily ligands comprise Bone Morphogenic Proteins (BMPs), Growth and Differentiation Factors (GDFs), anti-Mullerian hormone (AMH), Activin, Nodal and TGF-β. In general, Smad2 and Smad3 are phosphorylated by the ALK4, 5 and 7 receptors in the TGF-β/Activin pathway. In contrast, Smad1, Smad5 and Smad8 are phosphorylated as part of the bone morphogenetic protein (BMP) pathway. Although there is some cross-over between pathways, in the context of this invention, an activator of the TGF-β signaling pathway is preferably an activator of the TGF-β pathway which acts via Smad2 and Smad3.

According to certain embodiments, the activator of the TGF-β signaling pathway is selected from the group consisting of, but not limited to, Activin A, TGF-β, BMP2, BMP7, GDF9, NODAL, and any combination thereof. Each possibility represents a separate embodiment of the present invention. Any other activators of the TGF-β signaling pathway suitable in the method of the invention can be also applied.

According to certain exemplary embodiments, the activator of the TGF-β signaling pathway is Activin A.

Glycogen synthase kinase 3 (GSK3) is highly conserved from yeast to mammals. Mammals express two GSK3 isoforms, α (51 kDa) and β (47 kDa), which are encoded by distinct genes and share 97% amino acid sequence identity within their catalytic domains. However, their sequences differ significantly outside the kinase domain 2. Both GSK3 isoforms appear to be ubiquitously expressed, and they seem to be functionally redundant in some signaling pathways, including Wnt-β-catenin signaling, but they perform distinct functions in others. Numerous studies have pointed to an association of GSK3 dysregulation, particularly hyperactivation, with various pathological conditions, including diabetes mellitus, obesity, inflammation, neurological disorders, and tumorigenesis.

According to certain embodiments, the inhibitor of the GSK3 signaling pathway is selected from the group consisting of, but not limited to, CHIR 99021 (C22H18C12N8) or a salt thereof, SB 216763, LY2090314, TWS119, Tideglusib, GSK-3β inhibitor 1, GSK-3β inhibitor 2, GSK-3β inhibitor 3, AR-A014418, TDZD-8, Kenpaullone, GSK 3 Inhibitor IX, Cromolyn sodium, CHIR-98014, AZD1080, SB 415286, IM-12, 9-ING-41, Indirubin-3′-monoxime, 1-Azakenpaullone, BRD0705, AZD2858, CP21R7, BIO-acetoxime, Bikinin, VP3.15, VP3.15 dihydrobromide, GNF4877, KY19382, SAR502250, A 1070722, (R)-BRD3731, BRD3731, BIP-135, 5-Iodo-indirubin-3′-monoxime, BRD5648, GSK-3 inhibitor 1, GSK-3/CDK5/CDK2-IN-1, Indirubin-3′-monoxime-5-sulphonic acid, a GSK3β-inhibiting flavonoid, lithium, and any combination thereof. Each possibility represents a separate embodiment of the present invention. Any other GSK3-inhibitor suitable in the method of the invention can be also applied.

According to certain embodiments, the CHIR-99021 salt is selected from the group consisting of CHIR-99021 monohydrochloride and CHIR-99021 trihydrochloride. Each possibility represents a separate embodiment of the present invention.

According to certain exemplary embodiments, the inhibitor of the GSK3 signaling pathway is CHIR-99021 (6-[[2-[[4-(2,4-Dichlorophenyl)-5-(5-methyl-1H-imidazol-2-yl)-2-pyrimidinyl]amino]ethyl]amino]-3-pyridinecarbonitrile, C₂₂H₁₈C₁₂N₈).

Flavonoids were found to have inhibition activity towards GSK3β(e.g., Johnson L J, et al., 2011. J Med Food 14(4):325-333; Jung Y et al., 2017. Appl Biol Chem 60(3):227-232). According to certain embodiments, the inhibitor of the GSK3 signaling pathway according to the teachings of the present invention is a GSK3β-inhibiting flavonoid. According to some embodiments, the flavonoid is selected from the group consisting of luteolin, apigenin, quercetin, myricetin and any combination thereof. Each possibility represents a separate embodiment of the present invention.

According to certain exemplary embodiments, the culture medium for forming a plurality of skeletal muscle-committed progenitor cells comprises the combination of Actin A as an activator of the TGF-β signaling pathway and CHIR-99021 as an inhibitor of the GSK3 signaling pathway.

It will be understood by the skilled person that the concentration of an effective amount of the activator of the TGF-β signaling pathway and of the inhibitor of the GSK3 signaling pathway will depend on the specific type of the agent used. Typically, the culture medium for forming a plurality of skeletal muscle-committed progenitor cells is devoid of growth factors other than growth factors activating the TGF-β signaling pathway and growth factors inhibiting the GSK3 signaling pathway. In alternative embodiments, such additional growth factors may be added to the medium for a limited time at certain growth stages of the cells.

According to certain embodiments, the culture medium is serum free. As used herein, the term “serum-free” with regard to a medium refers to a medium with no animal sera.

According to certain embodiments, the culture medium is animal-derived component-free. As used herein, the term “animal-derived component-free” with regard to a medium refers to a medium not containing any component of animal origin, particularly to a medium not containing mammal-derived components.

According to certain embodiments, culturing is performed under 3-dimensional (3D) culture conditions. A 3D cell culture is an artificially created environment in which biological cells are permitted to grow or interact with their surrounding cells. 3D cell culture allows the formation of self-assembled cell aggregate(s) or cluster(s) in in vitro growth mimicking earliest in vivo developmental steps. According to certain exemplary embodiments, the 3D culture comprises cells grown in a liquid suspension, typically within a vessel.

The terms “vessel” or “tissue culture vessel” are used herein interchangeably and refer to any receptacle in which the cells can grow in suspension. The receptacle can be of a variety of sizes, from the range of milliliters (e.g. non-adherent plate or Erlenmeyer flask) to the range of thousands of liters (e.g., bioreactor or culture bags).

According to certain embodiments, the suspension culture is devoid of adherent material and/or support matrix and the cells and/or cell clusters are freely suspended/floating within the liquid. According to these embodiments, the suspension culture is maintained in a vessel having walls of a material to which the cells do not adhere.

When the cells are grown in a suspension culture without any adherent material/support matrix the cells tend to group themselves with other cells to form aggregates of cells. The cell aggregates may be in the form of clusters, spheroids, organoids and the like.

According to certain embodiments, the culturing is performed under conditions forming adhered monolayers, also referred to herein as a “2-dimensional (2D) culture”. Any adherent substrate/support matrix known to be used for cell culturing can be used according to the teachings of the present invention. Examples include growth plate coated with adherent substance (e.g., inactivated feeder cells, an organic extracellular matrix like matrigel or vitronectin, or feeder cell conditioned medium), or hydrogels.

Pluripotent stem cells derived from any source as are known or will be known in the art can be used according to the teachings of the present invention. According to certain embodiments, the PSCs are derived from human. According to additional or alternative embodiments, the PSCs are derived from non-human animal. According to certain embodiments, the non-human animal is selected from the group consisting of ungulate, poultry, aquatic animals, invertebrate and reptiles. According to certain embodiments, the ungulate is selected from the group consisting of a bovine, a sheep, a goat, an equine, a pig, a giraffe, a camel, a deer, a hippopotamus, or a rhinoceros. Each possibility represents a separate embodiment of the present invention. According to certain exemplary embodiments, the ungulate is a bovine.

According to some embodiments, the PSCs are embryonic stem cells (ESCs).

According to some embodiments, the PSCs are non-embryonic stem cells (ESCs).

According to certain embodiments, the PSCs are induced PSCs (iPSCs) reprogrammed from somatic cells.

According to certain embodiments, the PSCs are induced PSCs (iPSCs) reprogrammed from somatic cells not including ESCs.

The reprogramming of cells to produce iPSCs can be performed by any method known in the art, including, for example, the method described in Bessi et al., 2021. Cells 10(6):1531; Kawaguchi et al., 2015. PLoS One 10(8):e0135403; Zhao et al., 2021. PNAS 118 (15):e2018505118; Poleganov et al., Hum. Gene Ther. 2015; 26:751-766).

According to certain embodiments, isolation and/or culturing of the PSCs, particularly bovine PSCs, and/or reprogramming of cells to produce iPSCs can be performed by the methods described in International (PCT) Application Publication No. WO 2020/230138 to the Applicant of the present invention.

Commercial preparations of PSCs, for example of bovine-derived PSCs are also available, including blastocyst-derived PSCs.

According to certain embodiments, at the time of seeding, the medium further comprises an inhibitor of Rho-associated protein kinase (Rock). Any Rock inhibitor currently known in the Art or to be developed in the future can be used according to the teachings of the present invention. According to certain embodiments, the Rock inhibitor is selected from the group consisting of Thiazovivin, Fasudil, Ripasudil, Netarsudil, RKI-1447, Y-27632, GSK429286A, Y30141. Each possibility represents a separate embodiment of the present invention. According to certain exemplary embodiments, the Rock inhibitor is Y-27632 dihydrochloride (1R,4r)-4-((R)-1-aminoethyl)-N-(pyridin-4-yl)cyclohexanecarboxamide).

According to certain embodiments, culturing the plurality of PSCs is performed continuously in the medium comprising the combination of at least one activator of the TGF-β signaling pathway and at least one inhibitor of the GSK3 signaling pathway.

According to certain additional or alternative embodiments, culturing the plurality of PSCs is performed in cycles, wherein the medium comprising the combination of at least one activator of the TGF-β signaling pathway and at least one inhibitor of the GSK3 signaling pathway is replaced after each cycle. The combination of at least one activator of the TGF-β signaling pathway and at least one inhibitor of the GSK3 signaling pathway in each cycle may be the same or different.

According to certain embodiments, culturing is performed for a time period enabling to reach from about 10% to about 90% skeletal muscle-committed progenitor cells out of the total number of the plurality of cells.

According to certain embodiments, the time period enabling to reach from about 10% to about 90% skeletal muscle-committed progenitor cells out of the total number of the plurality of cells is from about 3 days to about 7 days. According to certain embodiments, the time period is from about 3 days to 6 days, 3 days to 5 days, or 3 days to 4 days. Each possibility represents a separate embodiment of the present invention. According to certain embodiments, the time period enabling to reach from about 10% to about 90% skeletal muscle-committed progenitor cells out of the total number of the plurality of cells is 4 days.

This short time frame of culturing, together with the option to use 3D culture, particularly in bioreactors, are a significant advantage of the methods of the invention over hitherto known methods for producing skeletal muscle-committed progenitor cells, enabling use of the methods in large-scale production facilities, and particularly their use in the food industry for producing cultured meat product, where costs must be reduced to enable replacement of slaughter meat.

According to certain embodiments, the present invention provides a plurality of cells comprising skeletal muscle-committed progenitor cells produced as described hereinabove.

According to certain embodiments, the plurality of cells comprises from about 10% to about 90% skeletal muscle-committed progenitor cells out of the total number of cells. According to some embodiments, the plurality of cells comprises from about 15% to about 90%, about 25% to about 90%, about 30% to about 90%, about 40% to about 90%, about 45% to about 90%, or about 50% to about 90%, skeletal muscle-committed progenitor cells out of the total number of cells.

According to yet further certain embodiments, the plurality of cells further comprises at least one additional lineage-committed progenitor cells. According to certain embodiments, the lineage-committed progenitor cells are selected from the group consisting of stromal-committed progenitor cells, adipocyte-committed progenitor cells and a combination thereof. Each possibility represents a separate embodiment of the present invention.

According to certain embodiments, the plurality of cells comprises from about 10% to about 90% stromal-committed progenitor cells out of the total number of cells. According to certain embodiments, the plurality of cells comprises from about 10% to about 90% adipocyte-committed progenitor cells out of the total number of cells.

According to certain embodiments, the muscle committed progenitor cells produced by the method of the present invention comprise at least one mesodermal marker selected from the group consisting of TBXT, TBX6, MSGN1, Pax3 and any combination thereof and the at least one early myogenic marker is Six1. Each possibility represents a separate embodiment of the present invention. According to certain embodiments, the skeletal muscle-committed progenitor cells are characterized by the expression of MSGN1 and Six1. Each possibility represents a separate embodiment of the present invention.

According to certain embodiments, the plurality of cells comprising skeletal muscle-committed progenitor cells is essentially devoid if PSCs. According to some embodiments, the plurality of cells comprising skeletal muscle-committed progenitor cells is devoid if PSCs.

According to certain embodiments, the plurality of cells comprising skeletal muscle-committed progenitor cells is of non-human animal. According to these embodiments, the plurality of cells is produced from non-human-animal PSCs. The non-human animal is as described hereinabove. According to certain exemplary embodiments, the non-human animal is bovine.

According to certain embodiments, the plurality of cells comprising skeletal muscle-committed progenitor cells comprises at least one GSK3β inhibiting flavonoid or metabolites thereof. According to certain embodiments, the plurality of cells comprising skeletal muscle-committed progenitor cells comprises at least one flavonoid selected from the group consisting of luteolin, apigenin, quercetin, myricetin, metabolites thereof and any combination thereof. Each possibility represents a separate embodiment of the present invention. The amount of the at least one flavonoid or metabolites thereof may vary, from one to several nanomolar to hundreds micromolar. According to some embodiments, the amount of the at least one flavonoid or metabolite thereof is from 1 nM to 100 μM, or from 1 nM to 10 μM, or 1 nM to 1 μM. Each possibility represents a separate embodiment of the present invention.

According to certain aspects, the present invention provides a method of producing a plurality of differentiated cells comprising skeletal muscle cells, the method comprising:
a) culturing a plurality of pluripotent stem cell (PSC) in a culture medium comprising a combination of: (i) at least one activator of the TGF-beta (TGF-β) signaling pathway and (ii) at least one inhibitor of the GSK3 signaling pathway, thereby forming a plurality of cells comprising skeletal muscle-committed progenitor cells;
b) depositing the plurality of cells comprising skeletal muscle-committed progenitor cells obtained in step (a) on an adherent material and/or support matrix; and
c) culturing the plurality of cells in a differentiation medium promoting differentiation of the skeletal muscle-committed progenitor cells to skeletal muscle cells,
thereby producing a plurality of differentiated cells comprising skeletal muscle cells.

The PSCs, activator of the TGF-β signaling pathway, inhibitor of the GSK3 signaling pathway and the culture medium; and processes of steps (a) and (b) are as described herein above.

According to certain embodiments, the plurality of cultured PSCs is not genetically modified. The methods of the present invention enable producing the plurality of differentiated cells comprising skeletal muscle cells without performing any genetic manipulation throughout the entire process. According to these embodiments, the non-genetically engineered skeletal muscle cells and tissues comprising same may have an advantage in certain applications in the pharmaceutical and food industries.

According to certain alternative embodiments, the plurality of cultured PSCs is genetically modified. Employing genetic engineering methods may facilitate, for example, the formation of induced PSC (iPSCs) from somatic cells, having the advantage of not relying on embryonic stem cells, particularly without the need of embryonic mammalian stem cells.

Any method as is known or will be known in the art for depositing the plurality of cells comprising skeletal muscle committed progenitor cells on an adherent material and/or support matrix can be used with the teachings of the present invention.

According to certain embodiments, the adherent material is selected from the group consisting of inactivated feeder cells, an organic extracellular matrix like Matrigel or vitronectin, or feeder cell conditioned medium. Each possibility represents a separate embodiment of the present invention.

According to certain embodiments, the adherent material or support matrix is in a form selected from the group consisting of a nanocarrier, a microcarrier, a macro-carrier, a scaffold, a tissue-culture plate, a tissue culture vessel and the like. Each possibility represents a separate embodiment of the present invention.

According to certain embodiments, the support matrix is in a form selected from semi-solid form and solid form.

According to certain embodiments, the depositing is performed by bio-printing, using suitable printers as are known in the art. According to some embodiments, bio-printing is performed as described in International (PCT) Application Publication No. WO 2022/162662.

Any differentiation medium as is known or will be known in the art for facilitating the differentiation of muscle-committed progenitor cells to skeletal muscle cells can be used according to the teachings of the present invention. Advantageously, the present invention now discloses that a serum-free differentiation medium, furnished with nutrients including, for example vitamins, inorganic salts, amino acids, anti-oxidants, sugars etc. and certain hormones, for example Insulin, suffices for differentiation, without the need of a large-number of costly growth factors.

According to certain exemplary embodiments, the differentiation medium is devoid of an activator of the TGF-β signaling pathway and of an inhibitor the GSK3 signaling pathway. According to further exemplary embodiments, the differentiation medium is devoid of Activin A and CHIR 99021.

According to certain embodiments, the cells are cultured in the differentiation medium for from about 3 days to about 27 days. According to some embodiment, the cells are cultured in the differentiation medium for from about 3 days to about 26 days, to about 25 days, to about 24 days, to about 23 days, to about 22 days, to about 22 days, to about 20 days, to about 19 days, to about 18 days, to about 17 days, to about 16 days, to about 15 days, to about 14 days, to about 13 days, to about 12 days, to about 11 days, to about 10 days, to about 9 days, to about 8 days, to about 7 days, or to about 6 days. Each possibility represents a separate embodiment of the present invention. According to some embodiments, the cells are cultured in the differentiation medium for from about 3 days to about 15 days, or from about 5 days to about 10 days or from about 6 days to about 9 days. According to some embodiments, the cells are cultured in the differentiation medium for about 7 days.

As for the time required for obtaining the skeletal muscle-committed progenitor cells according to the invention, the time of differentiating the progenitor cells to muscle cells is also reduced compared to hitherto known methods. According to certain embodiments, the entire period for obtaining an engineered tissue comprising muscle cells is from about 6 days to about 30 days. According to certain embodiments, the entire period for obtaining an engineered tissue comprising muscle cells is from about 7 days to about 25 days, from about 8 days to about 24 days, from about 9 days to about 23 days, from about 10 days to about 22 days, from about 10 days to about 21 days, from about 10 days to about 21 days, from about 10 days to about 21 days, from about 10 days to about 20 days, from about 10 days to about 19 days, from about 10 days to about 18 days, from about 10 days to about 17 days, from about 10 days to about 16 days, or from about 10 days to about 15 days.

According to certain embodiments, the entire period for obtaining an engineered tissue comprising muscle cells is from about 11 days to about 14 days.

This overall time period of up to about 30 days, typically of 11-14 days is highly advantageous in large scale production of engineered tissues comprising muscle cells.

According to certain embodiments, the plurality of cells deposited on the adherent material and/or support matrix further comprises at least one additional lineage committed progenitor cells. According to some embodiments, the lineage-committed progenitor cells are selected from stromal-committed progenitor cells, adipocyte-committed progenitor cells and a combination thereof. Each possibility represents a separate embodiment of the present invention. According to these embodiments, the formed plurality of cells further comprises at least one of stromal cells and adipose cells.

According to some embodiments, the stromal cells are extracellular-matrix (ECM) producing cells.

According to certain embodiments, the method of the invention further comprises separating the plurality of differentiated cells comprising muscle cells and optionally at least one of stromal cells and adipocyte cells from the adherent material and/or support matrix.

According to certain embodiments, the present invention further provides a plurality of differentiated cells comprising differentiated skeletal muscle cells produced by the methods of the invention.

According to certain embodiments, the differentiated skeletal muscle cells produced by the methods of the invention are characterized by the expression of at least one myogenic marker is selected from the group consisting of Myf5, Pax7, MEF2C, SIX1, NYOD1, MYOG, MYH3, MYH7, NYH8, MB, MYMK and any combination thereof. Each possibility represents a separate embodiment of the present invention.

According to certain embodiments, the plurality of differentiated cells comprises from about 10% to about 90% skeletal muscle cells out of the total number of cells. According to some embodiments, the plurality of differentiated cells comprises from about 15% to about 90%, about 25% to about 90%, about 30% to about 90%, about 40% to about 90%, about 45% to about 90%, or about 50% to about 90%, skeletal muscle cells out of the total number of cells. According to certain embodiments, the plurality of differentiated cells comprises about 30% skeletal muscle cells out of the total number of cells. According to certain embodiments, the skeletal muscle cells are viable cells.

According to certain embodiments, the plurality of differentiated cells comprises from about 10% to about 90% stromal cells out of the total number of cells. According to some embodiments, the plurality of differentiated cells comprises from about 15% to about 90%, about 25% to about 90%, about 30% to about 90%, about 40% to about 90%, about 45% to about 90%, or about 50% to about 90%, stromal cells out of the total number of cells. According to certain embodiments, the stromal cells are viable cells.

According to certain embodiments, the plurality of differentiated cells comprises from about 10% to about 90% adipose cells out of the total number of cells. According to some embodiments, the plurality of differentiated cells comprises from about 15% to about 90%, about 25% to about 90%, about 30% to about 90%, about 40% to about 90%, about 45% to about 90%, or about 50% to about 90%, adipose cells out of the total number of cells. According to certain embodiments, the adipose cells are viable cells.

According to certain embodiment, the plurality of differentiated cells comprises detectable amount of at least one GSK3β inhibiting flavonoid or metabolites thereof as described hereinabove.

According to certain embodiments, the plurality of differentiated cells comprising muscle cells and optionally at least one of stromal cells and adipocyte cells forms an engineered tissue. According to certain embodiments, the engineered tissue comprises the adherent material and/or support matrix used in the production of the plurality of differentiated cells. According to certain additional or alternative embodiments, the engineered tissue is devoid of the adherent material and/or support matrix used in the production of the plurality of differentiated cells.

According to yet further certain aspects, the present invention provides a plurality of in vitro grown cells comprising skeletal muscle-committed progenitor cells, wherein the skeletal muscle-committed progenitor cells are characterized by the expression of at least one mesodermal marker and/or at least one early myogenic marker.

According to certain embodiments, the at least one mesodermal marker is selected from the group consisting of TBXT, TBX6, MSGN1, Pax3 and any combination thereof and the at least one early myogenic marker is Six1. Each possibility represents a separate embodiment of the present invention.

According to certain embodiments, the skeletal muscle-committed progenitor cells are characterized by the expression of MSGN1 and Six1. Each possibility represents a separate embodiment of the present invention.

According to certain embodiments, the plurality of in vitro grown cells comprising skeletal muscle-committed progenitor cells further comprises at least one additional lineage committed cells selected from the group consisting of stromal-committed progenitor cells, adipocyte-committed progenitor cells and a combination thereof. Each possibility represents a separate embodiment of the present invention.

According to certain embodiments, the plurality of in vitro grown cells comprising skeletal muscle-committed progenitor cells comprises at least one GSK3β inhibiting flavonoid and/or metabolites thereof. The flavonoids and mounts are as described hereinabove.

According to additional certain aspects, the present invention provides a plurality of in vitro grown differentiated cells comprising differentiated skeletal muscle cells, wherein the differentiated skeletal muscle cells are characterized by the expression of at least one myogenic marker.

According to certain embodiments, the at least one myogenic marker is selected from the group consisting of Myf5, Pax7, MEF2C, SIX1, NYOD1, MYOG, MYH3, MYH7, NYH8, MB, MYMK and any combination thereof. Each possibility represents a separate embodiment of the present invention.

According to certain embodiments, the plurality of in vitro grown differentiated cells further comprises at least one of stromal cells, adipocytes or a combination thereof. Each possibility represents a separate embodiment of the present invention.

According to certain embodiments, the stromal cells are extracellular-matrix (ECM) producing cells.

According to certain embodiments, the cells within the plurality of in vitro grown differentiated cells are non-human-animal cells.

The present invention further encompasses an engineered tissue comprising the plurality of in-vitro grown differentiated cells comprising skeletal muscle cells.

The muscle-committed progenitor cells, the plurality of differentiated cells and an engineered tissue comprising same produced by the methods of the invention are suitable for a variety of uses, including therapeutic applications and use as a food substance.

As described hereinabove, the media to be used, the time required for obtaining the plurality of differentiated cells comprising skeletal muscle cells and the overall efficacy, make the methods of the invention highly suitable for use in the food industry, particularly in technologies of producing cell cultured meat. Thus, the present invention specifically encompasses cultured meat and cultured meat product comprising the skeletal muscle cells and/or engineered tissue comprising same of the invention.

According to these embodiments, the engineered tissue or the cultured food product optionally further comprises at least one vegetable protein. According to certain embodiments, the engineered tissue and/or cultured meat further comprises at least one additional food-safe supplement. Suitable additional supplements include, but are not limited to, saturated and/or unsaturated fatty acids; lipids; flavoring agents; coloring agents; texturants; edible fibers; and the like. Each possibility represents a separate embodiment of the present invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### RNA Extraction & RT-PCR Analysis:

For gene expression analysis, RNA was extracted from the bovine pluripotent stem cells, bovine committed and the skeletal muscle cells using NucleoSpin® RNA purification kit (Macherey-Nagel) and the RNA concentration was determined using Nanodrop One C (ThermoFisher). The purified RNA was reverse-transcribed using ReverseAid First strand cDNA synthesis kit (ThermoFisher) using Thermal Cycler SimpliAmp device (ThermoFisher). The cDNA was subjected to RT-PCR analysis using TaqMan Fast Advanced Master Mix (ThermoFisher) and the subsequent list of primers listed in Table 1 using QuantStudio 5 Real time PCR device (ThermoFisher).

TABLE 1
Primers and probes used for pluripotency, commitment and myogenic
markers amplification

| | | SEQ ID |
|---|---|---|
| Marker | Primer Sequence/Catalog Number | No. |
| Oct4 | F: CTCCTAAAGCAGAAGAGGATCAC | 1 |
| | R: TGAGCTGCAAAGCCTCAA | 2 |
| | Probe: TTCAGCCAAACGACTATCTGCCGT/ | 3 |
| | (3IABKFQ) | |
| Nanog | F: GCAGAAATACCTCAGTCTCCAG | 4 |
| | R: CTATTCCTCGGCCAGTTGTT | 5 |
| | Probe: TTCTGGAACCAGGTCTTCACCTGC | 6 |
| TBXT | F: CGTCTCCTTCAGCAAAGTCAA | 7 |
| | R: GATGTGGATTCGAGGCTCATAC | 8 |
| | Probe: TGTAAGGAGTTCAACATGATCTGGCCC | 9 |
| MSGN1 | F: CTGCACACCCTCAGGAATTATC | 10 |
| | R: TCCGTGAGCTCTCCGATATAC | 11 |
| | Probe: TTCAGCGTCTGGATCTTGGTGAGC | 12 |
| TBX6 | F: GGGAGAATGGCCGAAACT | 13 |
| | R: GCATCTCCACTCCCATAGC | 14 |
| | Probe: TAAGAGGGAGCGAGATGCCCG | 15 |
| Pax3 | F: GATCCTTTGCAGGTATCAGGAG | 16 |
| | R: TCTCGAATTTCCCAGCTGAAC | 17 |
| | Probe: AGCAAGCCCAAGCAGGTGACAA | 18 |
| Myf5 | F: CTGCACACCCTCAGGAATTATC | 19 |
| | R: TCCGTGAGCTCTCCGATATAC | 20 |
| | Probe: TTCAGCGTCTGGATCTTGGTGAGC | 21 |
| Pax7 | F: GGCATGTTTAGCTGGGAGAT | 22 |
| | R: CTCCTTCTTCCCGAACTTGATT | 23 |
| | Probe: ACCGTGCCCTCAGTGAGTTC | 24 |
| Six1 | F primer: CCAGGTCAGCAACTGGTTTA | 25 |
| | R primer: GCTTGTTGGAGGAGGAGTTATT | 26 |
| | Probe: AGGCCAAGGAAAGGGAGAACACCG | 27 |
| Mef2C | F primer: GACAAGGTGCTGCTCAAGTA | 28 |
| | R primer: CTGTGACCTACTGAATCGTCTG | 29 |
| | Probe: CGTGGAGACGTTGAGAAAGAAGGGC | 30 |
| MyoD | F primer: CAGCGGCGAACACTACA | 31 |
| | R primer: CTGTAGTAAGTGCGGTCGTAG | 32 |
| | Probe: TTCCGACGGCATGATGGACTACAG | 33 |
| MyoG | F primer: CAGTGAATGCAGCTCCCATA | 34 |
| | R primer: GATTGTGGGCGTCTGTAGG | 35 |
| | Probe: CAACCCAGGGGATCATCTGCT | 36 |
| MyhC3 | Bt03258391_m1 (ThermoFisher) | |
| MyhC7 | Bt03224257_m1 (ThermoFisher) | |
| MyhC8 | Bt04290444_m1 (ThermoFisher) | |
| MRF4/Myf6 | Bt03251628_m1 (ThermoFisher) | |
| Myoglobin | Bt03211694_m1 (ThermoFisher) | |
| MYMK | Bt04314692_m1 (ThermoFisher) | |

### Immunofluorescence

Bovine skeletal muscle cells in 2D culture were subjected to immunofluorescence analysis as following: The cells were fixed using 4% Paraformaldehyde (PFA; Santa Cruz Biotechnology), permeabilized using 0.5% Triton X-100 (Sigma) and blocked using 5% BSA (MP biomedicals). The cells were incubated with primary antibody anti-Myosin MF-20 (DSHB) 1:75 followed by secondary Goat Anti-Mouse IgG H&L Alexa Fluor 594) antibody (Abcam ab150116) 1:500. Nuclei were stained using 0.5 ag/ml of DAPI (Sigma). The imaging was performed using EVOS FL Auto 2 Fluorescent Microscopy (ThermoFisher).

### Example 1: Production of a Plurality of Skeletal Muscle Committed Cells

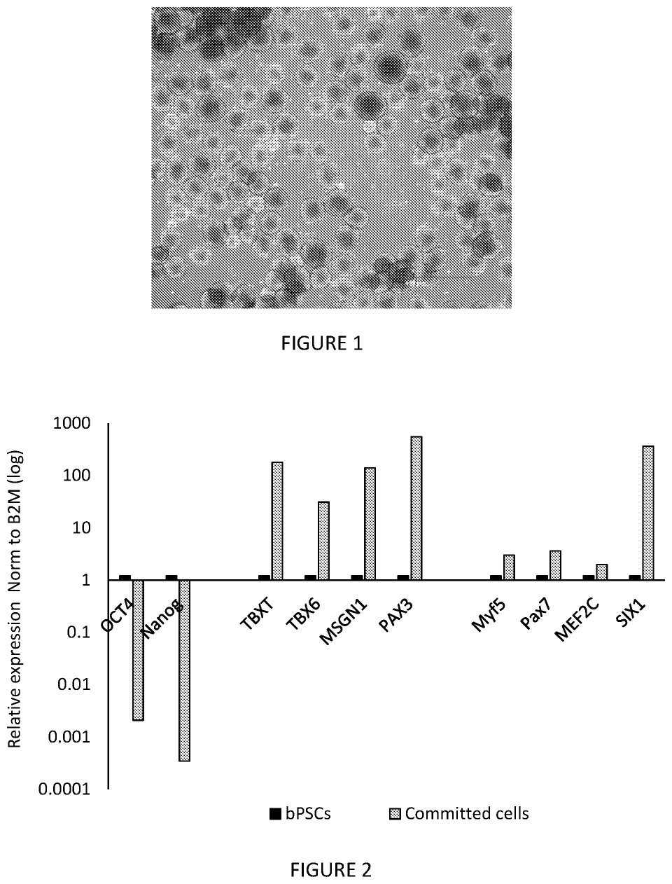

Bovine pluripotent stem cells (PSCs) were grown in 2D or 3D culture. The cells were harvested, dissociated, and resuspended in serum-free growth medium as a control or in serum-free growth medium containing Activin A (20 ng/ml) and CHIR-99021 (10 μM) (Skeletal Muscle Precursor medium) for 4 days. Rock Inhibitor (10 μM) was added during the growth of the first day under both the control and assay conditions. FIG. 1 shows a representative Bright Field image of skeletal muscle committed progenitor cells obtained from the bovine PSCs after 4 days.

After 4 days, the cells were harvested, and RT-PCR analysis was performed as described hereinabove to examine the expression of the mesodermal markers TBXT, TBX6, MSGN1 and Pax3; and of the early myogenic marker Six1.

As is shown in FIG. 2, a significant reduction in the expression of the pluripotent markers OCT4 and Nanog and a significant increase of the mesodermal markers TBXT, TBX6, MSGN1 and Pax3 as well as of the early myogenic marker Six1 (more than 10-fold compared to PSCs) was observed. Upregulation of fully differentiated muscle satellite markers, Myf5 and Pax7, was not significant at this early time point.

These results clearly demonstrate that culturing PSCs, particularly bovine PSCs in a basal serum-free medium supplemented with a combination of TGF-β activator (Activin A) and GSK3 inhibitor (CHIR-99021) induced differentiation processes towards skeletal muscle cells. After a relatively short time of about 4 days, the cells were expressing mesodermal markers and early myogenic markers at a level identifying the cells as skeletal muscle committed cells.

### Example 2: Differentiation of Bovine Pluripotent Stem Cells to Skeletal Muscle Cells

Bovine pluripotent stem cells (PSCs) were grown in 2D and/or 3D culture. The cells were dissociated, resuspended in serum-free growth medium containing Activin A (20 ng/ml) and CHIR99021 (10 μM) (Skeletal Muscle Precursor medium); in the same serum-free growth medium containing CHIR99021 (10 μM) only; or in the same serum-free growth medium containing TGFβ (2 ng/ml) and CHIR-99021 (10 μM), and cultured under suspension culture conditions. Rock Inhibitor (RI, 10 μM) was added to all media types.

The suspension culture conditions (“3D culture conditions”) include ultra-low adherent (ULA) 6-well plate (Corning) placed at 38.5° C. under 5% CO₂ and humidity of >75% under rotation. Medium was refreshed using the skeletal Muscle Precursor medium (without RI) and the formed aggregates were allowed to differentiate for a total of 4 days.

The aggregates comprising skeletal muscle committed cells were collected and seeded on in a tissue culture Vitronectin (0.005 mg/ml) pre-coated plate (“2D conditions”) in serum free growth medium. The plates were incubated at 38.5° C. under 5% CO₂ and humidity of >75% for additional 7 days (total process 11 days); medium was replaced on day 1 and day 3 (without RI).

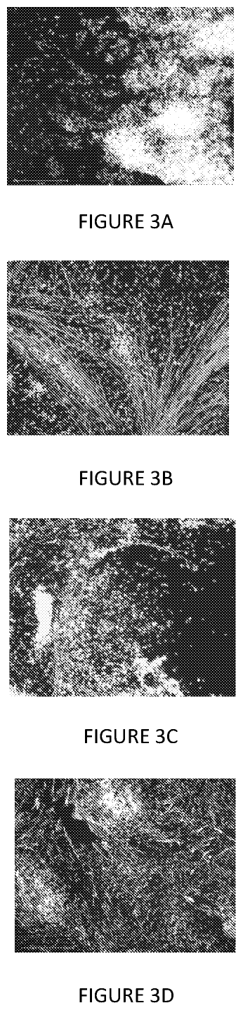

Representative immunofluorescence staining of Myosin heavy chain (using MF-20 antibody as described hereinabove) after total of 11 days of differentiation indicates the presence of skeletal muscle cells in a culture subjected to medium containing a combination of Activin A and CHIR-99021 (FIG. 3B), and to somewhat lesser extent in a culture subjected to medium containing the combination of TGFβ, additional activator of the TGF-beta (TGF-β) signaling pathway and CHIR-99021 (FIG. 3C). To the contrary, no Myosin positive cells have been observed in the culture subjected to a medium containing CHIR-99021 only (FIG. 3A).

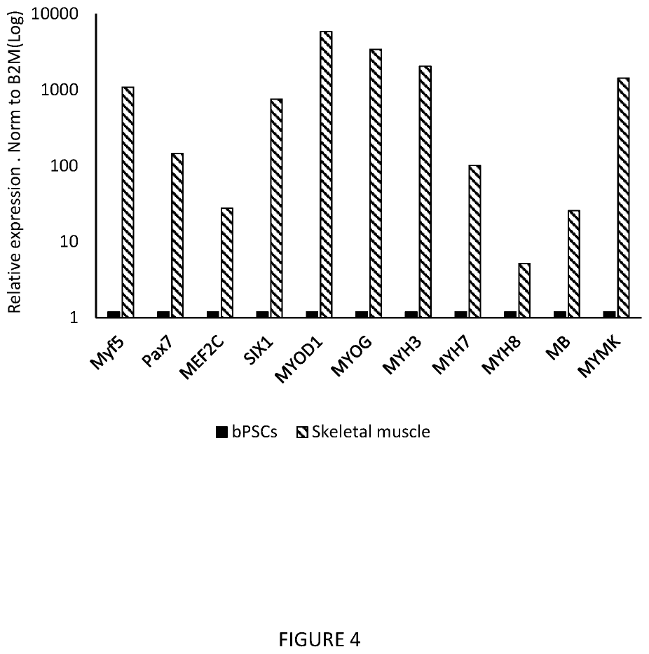

These results were further supported by RT-PCR analysis of the myogenic markers Myf5, Pax7, Mef2C, Six1, MyoD1, MyoG, MYH3, MYH7, MYH8, Myoglobin (MB) and Myomaker (MYMK) after total of 11 days of differentiation. All markers were up-regulated, indicating differentiation towards skeletal muscle (FIG. 4).

In additional experiments, PSCs were resuspended in in serum-free growth medium containing Activin A (20 ng/ml) and CHIR99021 (10 μM) and cultured in a 2D culture conditions, in tissue culture plate placed at 38.5° C. under 5% CO₂ and humidity of >75%. Medium was refreshed using the skeletal Muscle Precursor medium (without RI) and the formed aggregates were allowed to differentiate for a total of 4 days. After 4 days, the cells comprising skeletal muscle committed cells were collected and seeded in a 2D culture conditions as described for the aggregate obtained from 3D culture conditions described above. FIG. 3D shows that there is no significance difference between culturing the PSCs in 2D or 3D culture conditions at the commitment-induction stage with regard to the further differentiation of the committed cells to skeletal muscle cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of producing a plurality of cells comprising skeletal muscle-committed progenitor cells, the method comprising culturing a plurality of pluripotent stem cells (PSCs) in a culture medium comprising a combination of (i) at least one activator of the TGF-beta (TGF-β) signaling pathway and (ii) at least one inhibitor of the GSK3 signaling pathway; thereby producing a plurality of cells comprising skeletal muscle-committed progenitor cells.

2. (canceled)

3. The method of claim 1, wherein culturing is performed under three-dimensional (3D) culture conditions, wherein the 3D culture is selected from the group consisting of a 3D culture devoid of adherent material and/or support matrix and a 3D culture comprising at least one adherent material and/or support matrix.

4-7. (canceled)

8. The method of claim 1, wherein culturing is performed under two-dimensional (2D) culture conditions comprising at least one adherent material and/or support matrix.

9. The method of claim 1, wherein the at least one activator of the TGF-β signaling pathway is selected from the group consisting of: Activin A, TGF-β, BMP2, BMP7, GDF9, NODAL, and any combination thereof.

10. The method of claim 1, wherein the at least one inhibitor of the GSK3 signaling pathway is selected from the group consisting of: CHIR-99021 (C22H18C12N8) or a salt thereof, SB 216763, LY2090314, TWS119, Tideglusib, GSK-3β inhibitor 1, GSK-3β inhibitor 2, GSK-30 inhibitor 3, AR-A014418, TDZD-8, Kenpaullone, GSK 3 Inhibitor IX, Cromolyn sodium, CHIR-98014, AZD1080, SB 415286, IM-12, 9-ING-41, Indirubin-3′-monoxime, 1-Azakenpaullone, BRD0705, AZD2858, CP21R7, BIO-acetoxime, Bikinin, VP3.15, VP3.15 dihydrobromide, GNF4877, KY19382, SAR502250, A 1070722, (R)-BRD3731, BRD3731, BIP-135, 5-Iodo-indirubin-3′-monoxime, BRD5648, GSK-3 inhibitor 1, GSK-3/CDK5/CDK2-IN-1, Indirubin-3′-monoxime-5-sulphonic acid, a GSK3β-inhibiting flavonoid, lithium, and any combination thereof.

11-14. (canceled)

15. The method of claim 1, wherein the combination comprises Activin A and CHIR-99021

16-17. (canceled)

18. The method of claim 1, wherein at least one exist: (i) culturing the plurality of PSCs is performed continuously in the medium comprising the combination of at least one activator of the TGF-β signaling pathway and at least one inhibitor of the GSK3 signaling pathway; and (ii) culturing the plurality of PSCs is performed in cycles, wherein the medium comprising the combination of at least one activator of the TGF-β signaling pathway and at least one inhibitor of the GSK3 signaling pathway is replaced after each cycle.

19-20. (canceled)

21. The method of claim 1, wherein the produced plurality of cells further comprises at least one additional lineage committed progenitor cells.

22-23. (canceled)

24. The method of claim 1, wherein the PSCs are of an origin selected from the group consisting of non-human animal and human.

25-26. (canceled)

27. A method of producing a plurality of differentiated cells comprising skeletal muscle cells, the method comprising:
a. depositing the plurality of cells comprising skeletal muscle committed progenitor cells produced by the method of claim 1 on an adherent material and/or support matrix; and
b. culturing the plurality of cells in a differentiation medium promoting differentiation of the skeletal muscle-committed progenitor cells to skeletal muscle cells,
thereby producing a plurality of differentiated cells comprising skeletal muscle cells.

28. The method of claim 24, wherein the differentiation medium is devoid of activators of the TGF-β signaling pathway and of inhibitors of the GSK3 signaling pathway.

29. (canceled)

30. The method of claim 27, wherein the entire period for obtaining the plurality of differentiated cells comprising skeletal muscle cells from PSCs is from about 6 days to about 30 days.

31. (canceled)

32. The method of claim 27, wherein the plurality of differentiated cells further comprises at least one additional cell type selected from the group consisting of stromal cells and adipocytes.

33-34. (canceled)

35. A plurality of cells comprising skeletal muscle-committed progenitor cells produced by the method of claim 1.

36-37. (canceled)

38. The plurality of cells of claim 35, wherein the skeletal muscle-committed progenitor cells are produced from non-human animal PSCs.

39. (canceled)

40. A plurality of differentiated cells comprising skeletal muscle cells produced by the method of claim 27.

41. (canceled)

42. The plurality of differentiated cells of claim 40, wherein said plurality of differentiated cells further comprises at least one type of cells selected from the group consisting of stromal cells, adipocyte cells, and a combination thereof.

43. The plurality of differentiated cells of claim 42, wherein the stromal cells comprise collagen-producing cells.

44. An engineered tissue comprising the plurality of cells of claim 40.

45. A cultured food product comprising the plurality of differentiated cells of claim 40 and/or an engineered tissue comprising same.

46. The cultured food product of claim 45, wherein said cultured food product is cultured meat.

47. A plurality of in vitro grown cells comprising skeletal muscle-committed progenitor cells, wherein the skeletal muscle-committed progenitor cells are characterized by the expression of at least one mesodermal marker and/or at least one early myogenic marker.

48-50. (canceled)

51. The plurality of in vitro grown cells of claim 47, wherein said plurality of cells comprises at least one GSK3β inhibiting flavonoid and/or a metabolite thereof.

52. A plurality of in vitro grown differentiated cells comprising skeletal muscle cells, wherein the skeletal muscle cells are characterized by the expression of at least one myogenic marker.

53. (canceled)

54. The plurality of in vitro grown differentiated cells of claim 52, wherein said plurality of cells further comprises at least one of stromal cells, adipocytes or a combination thereof.

55. (canceled)

56. The plurality of in vitro grown differentiated cells of claim 47, wherein the cells are non-human-animal cells.

57. An engineered tissue comprising the plurality of in vitro grown differentiated cells of claim 52.

58. A cultured food product comprising the plurality of in vitro grown differentiated cells of claim 56 and/or an engineered tissue comprising same.

59. (canceled)
